(19) United States
(12) Patent Application Publication
(10) Pub. No.: US 2007/0129409 A1
(43) Pub. Date: Jun. 7, 2007

(54) Androgen receptor modulators

(76) Inventors: Lain-Yen Hu, Ann Arbor, MI (US); Daniel Y. Du, Milan, MI (US); Huangshu Lei, Waltham, MA (US); Wen-Song Yue, Ann Arbor, MI (US)

Correspondence Address: WARNER-LAMBERT COMPANY, 2800 PLYMOUTH RD, ANN ARBOR, MI, 48105, US

(21) Appl. No.: 10/580,078

(22) PCT Filed: Nov. 8, 2004
(86) PCT No.: PCT/IB04/03668

§ 371 Date: Jan. 10, 2007

Related U.S. Application Data
(60) Provisional application No. 60/523,885, filed on Nov. 20, 2003.

(51) Int. Cl. C07D 213/84; A61K 31/44

(52) U.S. Cl. 514/344; 546/289

(57) ABSTRACT

The present invention is directed to a new class of 5-cyano-2-amino pyridines and to their use as androgen receptor modulators. A further aspect of the invention is directed to a new animal model for finding compounds capable of alleviating androgenic alopecia.

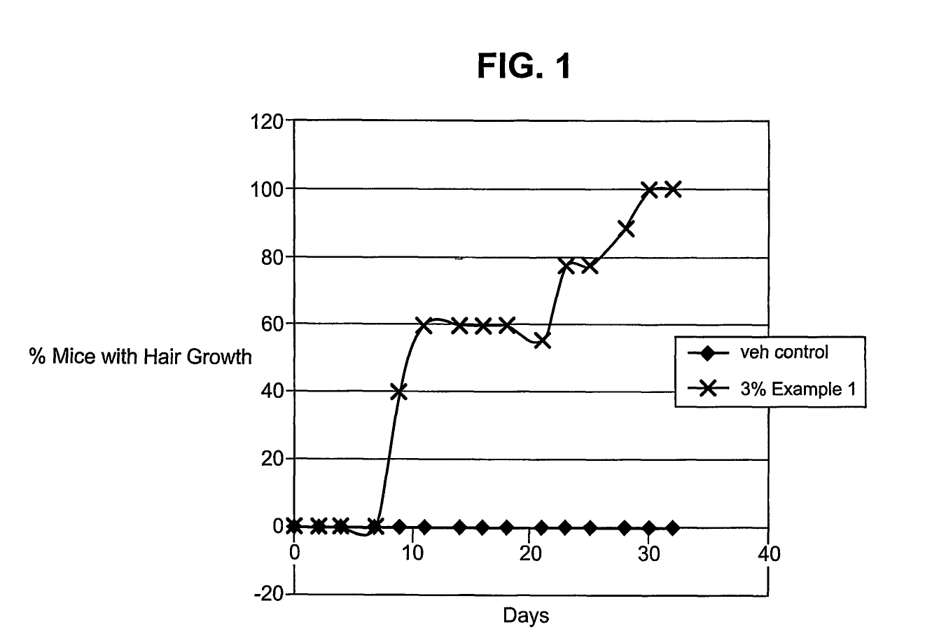

### FIELD OF THE INVENTION

The present invention is directed to a new class of pyridine derivatives and to their use as androgen modulators.

### BACKGROUND OF THE INVENTION

Alopecia, or balding, is a common problem which medical science has yet to cure. The physiological mechanism by which this hair loss occurs is not known. However, it is known that hair growth is altered in individuals afflicted with alopecia. Hair follicles undergo cycles of activity involving periods of growth, rest, and shedding. The human scalp typically contains from 100,000 to 350,000 hair fibers or shafts, which undergo metamorphosis in three distinct stages:
(a) during the growth phase (anagen) the follicle (i.e. the hair root) penetrates deep into the dermis with the cells of the follicle dividing rapidly and differentiating in the process of synthesizing keratin, the predominant component of hair. In non-balding humans, this growth phase lasts from one to five years;
(b) the transitional phase (catagen) is marked by the cessation of mitosis and lasts from two to several weeks; and
(c) the resting phase (telogen) in which the hair is retained within the scalp for up to 12 weeks, until it is displaced by new follicular growth from the scalp below.

In humans, this growth cycle is not synchronized. An individual will have thousands of follicles in each of these three phases. However, most of the hair follicles will be in the anagen phase. In healthy young adults, the anagen to telogen ratio can be as high as 9 to 1. In individuals with alopecia, this ratio can be reduced to as low as 2:1.

Androgenetic alopecia arises from activation of an inherited sensitivity to circulating androgenic hormones. It is the most common type of alopecia. It affects both men (50%) and women (30%), primarily of Caucasian origin. Gradual changes in the diameter and length of the hair shaft are experienced over time and with increasing age. Terminal hair is gradually converted to short, wispy, colorless vellus hair. As a consequence, men in their 20's and women in their 30's and 40's begin to notice their hair becoming finer and shorter. In males, most of the hair loss occurs at the front and vertex of the head. Females experience a thinning over their entire scalp. As discussed above, the anagen to telogen ratio is reduced significantly, resulting in less hair growth.

Minoxidil, a potassium channel opener, promotes hair growth. Minoxidil is available commercially in the United States under the trade name Rogaine®. While the exact mechanism of action of minoxidil is unknown, its impact on the hair growth cycle is well documented. Minoxidil promotes the growth of the hair follicle and increase the period of time that the hair follicle is in the anagen phase (i.e. increases the anagen to telogen ratio).

While minoxidil promotes hair growth, the cosmetic efficacy of this growth can vary widely. For example, Roenigk reported the results of a clinical trial involving 83 males who used a topical solution of 3% minoxidil for a period of 19 months. Hair growth occurred in 55% of the subjects. However, only 20% of the subjects considered the growth to be cosmetically relevant. (Clin. Res., 33, No. 4, 914A, 1985). Tosti reported cosmetically acceptable re-growth in 18.1% of his subjects. (Dermatologica, 173, No. 3, 136-138, 1986). Thus, the need exists in the art for compounds having the ability produce higher rates of cosmetically acceptable hair growth in patients with alopecia.

### SUMMARY OF THE INVENTION

In accordance with the present invention, a new class of 2-amino-5-cyanopyridines has been discovered. These compounds, and their pharmaceutically acceptable salts and hydrates thereof, may be represented by the following formula:

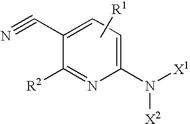

in which;
a) R¹ is represented by halogen, (C₁-C₂)alkyl, substituted with one or more halogens, or (C₁-C₂)alkoxy, substituted with one or more halogens,
b) R² is represented by hydrogen, or halogen,
c) X¹ and X² are each independently represented by:
   i) (C₁-C₁₂)alkyl, optionally substituted,
   ii) (C₂-C₁₂)alkenyl, optionally substituted,
   iii) (C₂-C₁₂)alkynyl, optionally substituted,
   iv) (C₃-C₁₀)cycloalkyl, optionally substituted,
   v) (C₃-C₁₀)cycloalkyl(C₁-C₆)alkyl, in which the alkyl and cycloalkyl moieties may each be optionally substituted,
   vi) (C₆-C₁₀)aryl, optionally substituted,
   vii) (C₆-C₁₀)aryl(C₁-C₆)alkyl, in which the alkyl and aryl moieties may each be optionally substituted,
   viii) —(CH₂)_{q}—CH₂—ZH, in which Z is S or O and q is an integer from 1-11,
   ix) —(CH₂)_{q}—Y—(CH₂)ₚ—CH₃, in which Y is O or S, n is an integer from 1 to 4, and p is an integer from 1 to 4;
   x) —[CH₂]ₘ—C(O)R³, in which m is an integer selected from 1 to 8, and R³ is represented by hydrogen, (C₁-C₁₂)alkyl, (C₆-C₁₀)aryl, or (C₆-C₁₀)aryl(C₁-C₆)alkyl, in which the alkyl and aryl moieties may each be optionally substituted,
   xi) —[CH₂]ₘ—C(O)—O—R⁴, in which m is as defined above and R⁴ is represented by hydrogen, (C₁-C₁₂)alkyl, (C₆-C₁₀)aryl, or (C₆-C₁₀)aryl(C₁-C₆)alkyl, in which the alkyl and aryl moieties may be optionally substituted,
   xii) —[CH₂]ₘ—C(O)—NR⁵R⁶ in which m is as described above, and R⁵ and R⁶ are each independently represented by hydrogen, (C₁-C₁₂)alkyl, (C₆-C₁₀)aryl, or (C₆-C₁₀)aryl(C₁-C₆)alkyl, in which the alkyl and aryl moieties may be optionally substituted,
   xiii) heteroaryl, optionally substituted,
   xiv) heteroaryl(C₁-C₆)alkyl, in which the heteroaryl and alkyl moieties may each be optionally substituted,
   xv) heterocyclic, optionally substituted, or,
   xvi) heterocyclic(C₁-C₆)alkyl, in which the alkyl and heterocyclic moieties may each be substituted.

The compounds of Formula I are androgen receptor modulators. The compounds have affinity for the androgen receptor and will cause a biological effect by binding to the receptor. Typically, the compounds will act as antagonists, but in selected embodiments they will act as partial agonists, full agonists, or tissue selective modulators. As androgen receptor modulators, the compounds can be used to treat, or alleviate, conditions associated with inappropriate activation of the androgen receptor. Examples of such conditions include, but are not limited to, acne, excess sebum secretion, androgenic alopecia, hormone dependant cancers such as prostrate cancer, and hirsutism. Those compounds which are partial agonists, or full agonists, can be used to treat osteoporosis, hypogonadism, or to stimulate increases in muscle mass, especially in wasting diseases.

The invention is also directed to pharmaceutical compositions containing at least one of the compounds of Formula I, in an amount effective to modulate activation of the androgen receptor. In a further embodiment, the invention is directed to an article of manufacture containing a compound of Formula I, packaged for retail distribution, in association with instructions advising the consumer on how to use the compound to alleviate a condition associated with inappropriate activation of the androgen receptor. An additional embodiment is directed to the use of a compound of Formula I as a diagnostic agent to detect inappropriate activation of the androgen receptor.

In a further embodiment, the compounds of Formula I are used topically to induce and/or stimulate hair growth and/or to slow down hair loss. The compounds may also be used topically to allow excess sebum secretion and/or of acne.

In a further embodiment the compounds can be used in livestock such as cattle, pigs, chickens, fish, etc. The compounds will increase the growth rate, and enhance the lean meat to fat ratio in the animals, and improve feed efficiency.

### DETAILED DESCRIPTION OF THE INVENTION

The headings within this document are only being utilized expedite its review by the reader. They should not be construed as limiting the invention or claims in any manner.

Definitions and Exemplification

As used throughout this application, including the claims, the following terms have the meanings defined below, unless specifically indicated otherwise. The plural and singular should be treated as interchangeable, other than the indication of number:
a. “C₁-C₁₂ alkyl” refers to a branched or straight chained alkyl group containing from 1 to 12 carbon atoms, such as methyl, ethyl, n-propyl, isopropyl, n-butyl, isobutyl, hexyl, octyl, decyl, etc. Such an alkyl group may be optionally substituted, in which up to 3 hydrogen atoms are replaced by a substituent selected from the group consisting of halogen, hydroxy, and trifluoromethyl
b. “C₁-C₄ alkyl” refers to a branched or straight chained alkyl group containing from 1 to 4 carbon atoms, such as methyl, ethyl, n-propyl, isopropyl, n-butyl, isobutyl, etc. Such an alkyl group may be optionally substituted, in which up to 3 hydrogen atoms are replaced by a substituent selected from the group consisting of halogen, hydroxy, and trifluoromethyl.
c. “C₁-C₆ alkyl” refers to a branched or straight chained alkyl group containing from 1 to 6 carbon atoms, such as methyl, ethyl, n-propyl, isopropyl, n-butyl, isobutyl, pentyl, etc. Such an alkyl group may be optionally substituted, in which up to 3 hydrogen atoms are replaced by a substituent selected from the group consisting of halogen, hydroxy, and trifluoromethyl.
d. “halogen” refers to a chlorine, fluorine or bromine atom.
e. “C₁-C₂ alkyl substituted with one or more halogen atoms” refers to a straight chained alkyl group containing 1 or 2 carbon atoms, i.e. methyl or ethyl, in which at least one hydrogen atom is replaced with a halogen. Examples include chloromethyl, difluoromethyl, trifluoromethyl, etc.
f. “C₁-C₆ alkoxy” refers to a straight or branched chain alkoxy group containing from 1 to 6 carbon atoms, such as methoxy, ethoxy, n-propoxy, isopropoxy, n-butoxy, isobutoxy, pentoxy, etc.
g. “C₂-C₁₂ alkenyl” refers to a straight-chain or branched-chain hydrocarbon radical containing from 2 to 12 carbon atoms and 1, or more, carbon-carbon double bonds. Examples of alkenyl radicals include ethenyl, propenyl, 1,4-butadienyl, 1-hexenyl, 1,3-octadienyl and the like. Such an alkenyl group may be optionally substituted, in which up to 3 hydrogen atoms are replaced by a substituent selected from the group consisting of halogen, hydroxy, and trifluoromethyl.
h. “C₂-C₁₂ alkynyl” refers to a straight-chain or branched-chain hydrocarbon radical containing from 2 to 12 carbon atoms and having 1, or more, carbon-carbon triple bonds. Examples of alkynyl radicals include ethynyl, propynyl, butynyl, octynyl, and the like. Such an alkynyl group may be optionally substituted, in which up to 3 hydrogen atoms are replaced by a substituent selected from the group consisting of halogen, hydroxy, and trifluoromethyl
i. “(C₆-C₁₀)aryl” means a cyclic, aromatic hydrocarbon containing from 6 to 10 carbon atoms. Examples of aryl groups include phenyl, naphthyl and biphenyl. Such an aryl moiety may be optionally substituted with up to 4 non-hydrogen substituents, each substituent is independently selected from the group consisting of halogen, nitrile, hydroxy, (C₁-C₆)alkyl, (C₁-C₆)alkoxy, (C₁-C₂)alkyl substituted with one or more halogens, (C₁-C₂)alkoxy substituted with one or more halogens, SR⁷, and NR⁷R⁸. R⁷ and R⁸ are each independently represented by C₁-C₆ alkyl or hydrogen. These substituents may be the same or different and may be located at any position of the ring, that is chemically permissible.
j. “(C₁-C₂)alkoxy substituted with one or more halogen atoms” refers to a straight chained alkoxy group containing 1 or 2 carbon atoms, i.e., methoxy or ethoxy in which at least one hydrogen atom is replaced with a halogen.
k. “heteroatom” includes oxygen, nitrogen, and sulfur.
l. “heteroaryl” refers to an aromatic ring having one, or more, heteroatoms selected from oxygen, nitrogen and sulfur. More specifically, it refers to a 5- or 6-, membered ring containing 1, 2, or 3 nitrogen atoms; 1 oxygen atom; 1 sulfur atom; 1 nitrogen and 1 sulfur atom; 1 nitrogen and 1 oxygen atom; 2 nitrogen atoms and 1 oxygen atom; or 2 nitrogen atoms and 1 sulfur atom. The 5-membered ring has 2 double bonds and the 6-membered ring has 3 double bonds. The term heteroaryl also includes bicyclic groups in which the heteroaryl ring is fused to a benzene ring, heterocyclic ring, a cycloalkyl ring, or another heteroaryl ring. Examples of such heteroaryl ring systems include, but are not limited to, pyrrolyl, furanyl, thienyl, imidazolyl, oxazolyl, indolyl, thiazolyl, pyrazolyl, pyridinyl, pyrimidinyl, purinyl, quinolinyl, and isoquinolinyl.
m. “heteroaryl, optionally substituted,” refers to a heteroaryl moiety as defined immediately above, in which up to 2 carbon atoms of the heteroaryl moiety may be substituted with a substituent, each substituent is independently selected from the group consisting of halogen, nitrile, hydroxy, (C₁-C₆)alkyl, (C₁-C₆)alkoxy, (C₁-C₂)alkyl substituted with one or more halogens, (C₁-C₂)alkoxy substituted with one or more halogens, SR⁷, and NR⁷R⁸.
n. “heterocycle” or “heterocyclic ring” refers to any 3- or 4-membered ring containing a heteroatom selected from oxygen, nitrogen and sulfur; or a 5-, 6-, 7-, 8-, 9-, or 10-membered ring containing 1, 2, or 3 nitrogen atoms; 1 oxygen atom; 1 sulfur atom; 1 nitrogen and 1 sulfur atom; 1 nitrogen and 1 oxygen atom; 2 oxygen atoms in non-adjacent positions; 1 oxygen and 1 sulfur atom in non-adjacent positions; or 2 sulfur atoms in non-adjacent positions. The 5-membered ring has 0 to 1 double bonds, the 6- and 7-membered rings have 0 to 2 double bonds, and the 8, 9, or 10 membered rings may have 0, 1, 2, or 3 double bonds. The term “heterocyclic” also includes bicyclic groups in which any of the above heterocyclic rings is fused to a benzene ring, a cyclohexane or cyclopentane ring or another heterocyclic ring (for example, indolyl, quinolyl, isoquinolyl, tetrahydroquinolyl, benzofuryl, dihydrobenzofuryl or benzothienyl and the like). Heterocyclics include: pyrrolidinyl, tetrahydrofuranyl, tetrahydrothiophenyl, piperidinyl, piperazinyl, azepane, azocane, morpholinyl, and quinolinyl.
o. “heterocyclic, optionally substituted” refers to a heterocyclic moiety as defined immediately above, in which up to 2 carbon atoms of the heterocycle moiety may be substituted with a substituent, each substituent is independently selected from the group consisting of halogen, nitrile, hydroxy, (C₁-C₆)alkyl, (C₁-C₆)alkoxy, (C₁-C₂)alkyl substituted with one or more halogens, (C₁-C₂)alkoxy substituted with 1 or more halogens, SR⁷, and NR⁷R⁸.
p. “C₃-C₁₀ cycloalkyl” refers to a saturated or partially saturated monocyclic, bicyclic or tricyclic alkyl radical wherein each cyclic moiety has 3 to 10 carbon atoms. Examples of cycloalkyl radicals include cyclopropyl, cyclobutyl, cyclopentyl, cyclohexyl, cyclooctyl, and the like. Such a cycloalkyl group may be optionally substituted, in which up to 3 hydrogen atoms are replaced by a substituent selected from the group consisting of halogen, nitrile hydroxy, (C₁-C₆)alkyl, (C₁-C₆)alkoxy, (C₁-C₂)alkyl substituted with one or more halogens, (C₁-C₂)alkoxy substituted with 1 or more halogens, SR⁷, and NR⁷R⁸.
q. “androgen” refers to testosterone and its precursors and metabolites, and 5-alpha reduced androgens, including but not limited to dihydrotestosterone. Androgen refers to androgens from the testis, adrenal gland, and ovaries, as well as all forms of natural, synthetic and substituted or modified androgens.
r. “pharmaceutically acceptable salts” is intended to refer to either pharmaceutically acceptable acid addition salts” or “pharmaceutically acceptable basic addition salts” depending upon actual structure of the compound.
s. “pharmaceutically acceptable acid addition salts” is intended to apply to any non-toxic organic or inorganic acid addition salt of the base compounds represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents, and which in comparison to their free base forms, generally demonstrate higher melting points.
t. “pharmaceutically acceptable basic addition salts” is intended to apply to any non-toxic organic or inorganic basic addition salts of the compounds represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
u. “prodrug” refers to compounds that are rapidly transformed in vivo to yield the parent compound of the above formulas, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.
v. “compound of Formula I”, “compounds of the invention”, and “compounds” are used interchangeably throughout the application and should be treated as synonoms.
w. “patient” refers to warm blooded animals such as, for example, guinea pigs, mice, rats, gerbils, cats, rabbits, dogs, monkeys, chimpanzees, and humans.
x. “treat” refers to the ability of the compounds to either relieve, alleviate, or slow the progression of the patient's disease (or condition) or any tissue damage associated with the disease.
y. “livestock” refers to animals suitable for human meat consumption. Examples include pigs, cattle, chickens, fish, turkeys, rabbits, etc.
z. “salts” is intended to refer pharmaceutically acceptable salts and to salts suitable for use in industrial processes, such as the preparation of the compound.

Some of the compounds of Formula I will exist as optical isomers. Any reference in this application to one of the compounds represented by Formula I is meant to encompass either a specific optical isomer or a mixture of optical isomers (unless it is expressly excluded). The specific optical isomers can be separated and recovered by techniques known in the art such as chromatography on chiral stationary phases or resolution via chiral salt formation and subsequent separation by selective crystallization. Alternatively utilization of a specific optical isomer as the starting material will produce the corresponding isomer as the final product.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

All of the compounds of Formula I contain a pyridine ring. To further exemplify the invention, the numbering system for this ring and its substitution pattern is shown below:

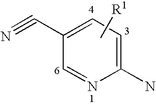

All of the compounds of this invention contain a pyridine ring that is substituted with at least 3 substituents. As depicted above, position 5 is substituted with a cyano group. Position 2 is substituted with a tertiary amine (i.e. neither X¹ nor X² can be hydrogen). Finally, the pyridine ring must be further substituted at the 3- or 4-position with a halogen, haloalkyl or haloalkoxy, as defined for R¹. Typically, R¹ will be trifluoromethyl and will be located at position 4 of the pyridine ring.

More specific embodiments of the invention are directed to compounds of Formula I in which:
i) R¹ is —CF₃ and is located at the 4-position of the pyridine ring, R² is H, X¹ and X² are each (C₁-C₁₂ alkyl),
ii) R¹ is —CF₃ and is located at the 4-position of the pyridine ring, R² is H, X¹ and X² are each (C₁-C₄ alkyl),
iii) R¹ is —CF₃ and is located at the 4-position of the pyridine ring, R² is H, X¹ is (C₁-C₄ alkyl) and X² is (C₆-C₁₀)aryl(C₁-C₆)alkyl,
iv) R¹ is —CF₃ and is located at the 4-position of the pyridine ring, R² is H, X¹ is (C₁-C₄ alkyl) and X² is phenyl (C₁-C₆)alkyl,
v) R′ is —CF₃ and is located at the 4-position of the pyridine ring, R² is H, X¹ is (C₁-C₄ alkyl) and X² is (C₃-C₁₀)cycloalkyl(C₁-C₆)alkyl, and,
vi) R¹ is —CF₃ and is located at the 4-position of the pyridine ring, R² is Cl and is located at the 5-position of the pyridine ring, X¹ and X² are each (C₁-C₄ alkyl.

More specific examples of compound encompassed by Formula I include:
i) (R)-(+)-6-[Methyl-(1-Phenyl-ethyl)-amino]-4-trifluoromethyl-nicotinonitrile,
ii) (R)-(+)-2-Chloro-6-[methyl-(1-phenyl-ethyl)-amino]-4-trifluoromethyl-nicotinonitrile,
iii) 6-[methyl-(1-phenyl-ethyl)-amino]-4-trifluoromethyl-nicotinonitrile,
iv) 6-[methyl-(1-phenyl-ethyl)-amino]-4-trifluoromethoxy-nicotinonitrile,
v) 6-[methyl-(1-(4-fluorophenyl)-ethyl)-amino]-4-trifluoromethyl-nicotinonitrile,
vi) 6-[methyl-(1-(3-hydroxyphenyl)-ethyl)-amino]-4-trifluoromethyl-nicotinonitrile,
vii) 6-[butyl(1-(3-hydroxyphenyl)-ethyl)-amino]-4-trifluoromethoxy-nicotinonitrile,
viii) 6-dipropylamino-4-trifluoromethyl-nicotinonitrile,
ix) 2-chloro-6-dimethylamino-4-trifluoromethyl-nicotinonitrile,
x) 6-(hexyl-octyl-amino)-4-trifluoromethyl-nicotinonitrile,
xi) 6-(sec-butyl-methyl-amino)-4-trifluoromethyl-nicotinonitrile,
xii) 6-[butyl-(2-hydroxy-ethyl)-amino]-4-trifluoromethyl-nicotinonitrile,
xiii) 6-(butyl-methyl-amino)-4-trifluoromethyl-nicotinonitrile,
xiv) 6-(benzyl-methyl-amino)-4-trifluoromethyl-nicotinonitrile,
xv) 6-(cyclohexyl-propyl-amino)-4-trifluoromethyl-nicotinonitrile,
xvi) 6-(cyclopropylmethyl-propyl-amino)-4-trifluoromethyl-nicotinonitrile,
xvii) 6-(sec-butyl-methyl-amino)-2-chloro-4-trifluoromethyl-nicotinonitrile,
xviii) 6-Dipropylamino-2-chloro-4-trifluoromethyl-nicotinonitrile,
xix) 6-(propyl-methyl-amino)-2-chloro-4-trifluoromethyl-nicotinonitrile, and,
xx) 6-(Butyl-methyl-amino)-2-chloro-4-trifluoromethyl-nicotinonitrile.
   Synthesis

The compounds of Formula I can be prepared using methods analogous to those known in the art for the preparation of amines. The reader's attention is directed to the Journal of Medicinal Chemistry, 1983, 26, 538-544 for a more detailed discussion. Scheme I below provides a general overview:

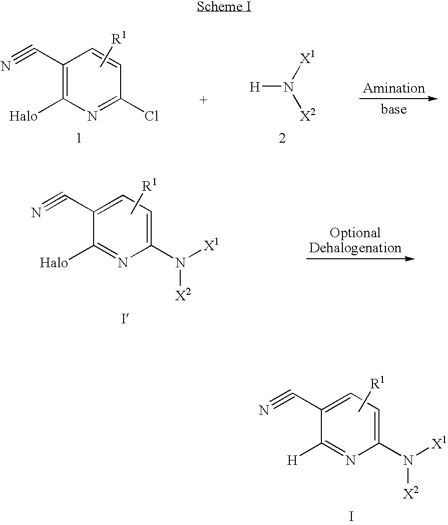

As depicted in Scheme I, the starting materials are a pyridine as depicted by structure 1 and an amine as depicted by structure 2. The pyridine as depicted by structure 1 is substituted with a chlorine at position 2, a cyano at position 5 and a halogen at position 6. R¹ should be represented by the same substituent as desired in the final product. Such compounds are commercially available from sources or alternatively can be prepared as known in the art. In the secondary amine represented by structure 2, X¹ and X² should be represented by the same substituents as desired in the final product. Such compounds are available from commercial sources or can be prepared as is known in the art.

As shown above, approximately equimolar amounts of the amine of structure 2 and the pyridine of structure 1 are contacted in the presence of a weak base, such as K₂CO₃, in an aprotic solvent such as dimethylformamide. The reactants are heated to a temperature of at least 70° C. and the reaction is allowed to proceed to completion. The desired product of Formula I′ can be recovered by extraction, evaporation, or other techniques known in the art. If desired, the compound can be further purified by flash chromatography, or other suitable techniques known in the art. If R² is to be represented by a halogen, then the synthesis is complete.

If R² is to be represented by hydrogen, it is necessary to subject the structure of Formula I′ to a dehalogenation reaction, as depicted. The dehalogenation is accomplished by contacting the compound of Formula I′ with a palladium catalyst in the presence of hydrogen and a weak base, such as triethyl amine. The reduction is typically carried out in an inert solvent, such as tetrahydrofuran, at elevated pressures, as is known in the art. A more detailed discussion of such reductions may be found at Journal of Medicinal Chemistry, 1987, 30, 2270-2277. The desired product of Formula I may then be purified by chromatography or other techniques known in the art

Medical and Cosmetic Uses

The compounds of Formula I are androgen receptor modulators. They can be used to alleviate any condition associated with inappropriate activation of the androgen receptor. Compounds acting as androgen antagonists may be used to treat, or alleviate, hormone dependent cancers such as prostate carcinomas, benign hyperplasia of the prostate, acne, hirsutism, excess sebum, alopecia, hypertrichosis, precocious puberty, prostamegaly, virilization, and polycystic ovary syndrome. Compounds acting as partial agonists, or full agonists, may be used to treat, or alleviate, male hypogonadism, male sexual dysfunction (impotence, male dysspemtatogenic sterility), abnormal sex differentiation (male hermaphroditism), male delayed puberty, male infertility, aplastic anemia, hemolytic anemia, sickle cell anemia, idiopathic thrombocytopenic purpura, myelofibrosis, renal anemia, wasting diseases (post operative, malignant tumor, trauma, chronic renal disease, burn or AIDS induced), abatement of pain in terminal carcinoma of female genitalia, inoperable breast cancer, mastopathy, endometriosis, female sexual dysfunction, osteoporosis, wound healing and muscle tissue repair.

In order to exhibit the therapeutic properties described above, the compounds need to be administered in a quantity sufficient to modulate activation of the androgen receptor. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the present invention may be administered by a variety of routes. They are effective if administered orally. The compounds may also be administered parenterally (i.e. subcutaneously, intravenously, intramuscularly, intraperitoneally, or intrathecally), rectally, or topically.

In a typical embodiment, the compounds are administered topically. Topical administration is especially appropriate for hirsutism, alopecia, acne and excess sebum. The dose will vary, but as a general guideline, the compound will be present in a dermatologically acceptable carrier in an amount of from 0.001 to 50 w/w %, and more typically from 0.1 to 10 w/w %. The dermatological preparation will be applied to the affected area from 1 to 4 times daily. “Dermatologically acceptable” refers to a carrier which may be applied to the skin or hair, and which will allow the drug to diffuse to the site of action. More specifically, it refers the site where modulation of activation of an androgen receptor is desired.

In a further embodiment, the compounds are used topically to relieve alopecia, especially androgenic alopecia. Androgens have a profound effect on both hair growth and hair loss. In most body sites, such as the beard and pubic skin, androgens stimulate hair growth by prolonging the growth phase of the hair cycle (anagen) and increasing follicle size. Hair growth on the scalp does not require androgens but, paradoxically, androgens are necessary for balding on the scalp in genetically predisposed individuals (androgenic alopecia) where there is a progressive decline in the duration of anagen and in hair follicle size. Androgenic alopecia is also common in women where it usually present as a diffuse hair loss rather than showing the patterning seen in men.

While the compounds will most typically be used to alleviate androgenic alopecia, the invention is not limited to this specific condition. The compounds may be used to alleviate any type of alopecia. Examples of non-androgenic alopecia include alopecia areata, alopecia due to radiotherapy or chemotherapy, scarring alopecia, stress related alopecia, etc.

As used in this application, “alopecia” refers to partial or complete hair loss on the scalp. The compounds will typically be used to alleviate androgenic alopecia. This condition afflicts both men and women. In males, the hair loss begins in the lateral frontal areas or over the vertex. For females, it is typically associated with thinning of the hair in the frontal and parietal regions. Complete hair loss in females is rare.

Thus, the compounds can be applied topically to the scalp and hair to prevent, or alleviate balding. Further, the compound can be applied topically in order to induce or promote the growth of hair on the scalp.

In a further embodiment of the invention, a compound of Formula I is applied topically in order to prevent the growth of hair in areas where such hair growth is not desired. One such use will be to alleviate hirsutism. Hirsutism is excessive hair growth in areas that typically do not have hair (i.e. a female face). Such inappropriate hair growth occurs most commonly in women and is frequently seen at menopause. The topical administration of the compounds will alleviate this condition leading to a reduction, or elimination of this inappropriate, or undesired, hair growth.

The compounds may also be used topically to decrease sebum production. Sebum is composed of triglycerides, wax esters, fatty acids, sterol esters and squalene. Sebum is produced in the acinar cells of the sebaceous glands and accumulates as these cells age. At maturation, the acinar cells lyse, releasing sebum into the lumenal duct so that it may be deposited on the surface of the skin.

In some individuals, an excessive quantity of sebum is secreted onto the skin. This can have a number of adverse consequences. It can exacerbate acne, since sebum is the primary food source for Propionbacterium acnes, the causative agent of acne. It can cause the skin to have a greasy appearance, typically considered cosmetically unappealing.

Formation of sebum is regulated by growth factors and a variety of hormones including androgen. The cellular and molecular mechanism by which androgens exert their influence on the sebaceous gland has not been fully elucidated. However, clinical experience documents the impact androgens have on sebum production. Sebum production is significantly increased during puberty, when androgen levels are their highest. Anti-androgens, such as finasteride, have been shown to decrease sebum secretion. For additional information on sebum production and androgens role in skin metabolism, see Moshell et al, Progress in Dermatology, vol. 37, No. 4, December 2003.

Thus, the compounds of formula I inhibit the secretion of sebum and thus reduce the amount of sebum on the surface of the skin. The compounds can be used to treat a variety of dermal diseases such as acne or seborrheic dermatitis.

In addition to treating diseases associated with excess sebum production; the compounds can also be used to achieve a cosmetic effect. Some consumers believe that they are afflicted with overactive sebaceous glands. They feel that their skin is oily and thus unattractive. These individuals can utilize the compounds of Formula I to decrease the amount of sebum on their skin. Decreasing the secretion of sebum will alleviate oily skin in individuals afflicted with such conditions.

In a further embodiment, those compounds acting as partial agonists, or full agonists, may be used to treat, or alleviate, osteoporosis. Osteoporosis is characterized by bone loss, resulting from an imbalance between bone resorption (destruction) and bone formation, which starts in the fourth decade and continues throughout life at the rate of about 1-4% per year (Eastell, Treatment of postmenopausal osteoporosis, New Eng. J. Med. 338: 736, 1998). In the United States, there are currently about 20 million people with detectable fractures of the vertebrae due to osteoporosis. In addition, there are about 250,000 hip fractures per year due to osteoporosis, associated with a 12%-20% mortality rate within the first two years, while 30% of patients require nursing home care after the fracture and many never become fully ambulatory again. In postmenopausal women, estrogen deficiency leads to increased bone resorption resulting in bone loss in the vertebrae of around 5% per year, immediately following menopause. Thus, first line treatment/prevention of this condition is inhibition of bone resorption by bisphosphonates, estrogens, selective estrogen receptor modulators (SERMs) and calcitonin. However, inhibitors of bone resorption are not sufficient to restore bone mass for patients who have already lost a significant amount of bone. The increase in spinal BMD attained by bisphosphonate treatment can reach 11% after 7 years of treatment with alendronate. In addition, as the rate of bone turnover differs from site to site; higher in the trabecular bone of the vertebrae than in the cortex of the long bones, the bone resorption inhibitors are less effective in increasing hip BMD and preventing hip fracture. Therefore, osteoanabolic agents, which increase cortical/periosteal bone formation and bone mass of long bones, would address an unmet need in the treatment of osteoporosis especially for patients with high risk of hip fractures.

A number of studies demonstrate that androgens are osteoanabolic in women and men. Anabolic steroids, such as nandrolone decanoate or stanozolol, have been shown to increase bone mass in postmenopausal women. Beneficial effects of androgens on bone in post-menopausal osteoporosis are well documented in recent studies using combined testosterone and estrogen administration (Hofbauer, et al., Androgen effects on bone metabolism: recent progress and controversies, Eur. J. Endocrinol. 140, 271-286, 1999). Thus those compounds of Formula I exhibiting agonist or partial agonist activity may be used to treat, or alleviate, osteoporosis, including primary osteoporosis such as senile, postmenopausal and juvenile osteoporosis, as well as secondary osteoporosis, such as osteoporosis due to hyperthyroidism or Cushing syndrome (due to corticosteroid treatment), acromegaly, hypogonadism, dysosteogenesis and hypophosphatasemia. Other bone related indications amendable to treat from androgen agonists include osteoporotic fracture, childhood idiopathic bone loss, alveolar bone loss, mandibular bone loss, bone fracture, osteotomy, periodontitis, or prosthetic ingrowth.

Those compounds acting as agonists, or partial agonists, can also be used to stimulate muscle mass in patients afflicted with wasting diseases, such as AIDS, cancer cachexia, burns, renal disease, etc. Patients suffering from trauma, bedsores, age, etc. can also benefits from the anabolic effects of androgens.

Co-Administration

In a further embodiment of the invention, the compounds of Formula I can be co-administered with other compounds to further enhance their activity, or to minimize potential side effects. For example, potassium channel openers, such as minoxidil, are known to stimulate hair growth and to induce anagen. Examples of other potassium channel openers include (3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran, diaxozide, and P1075 which is under development by Leo Pharmaceuticals. Such compounds can be co-administered with the compounds of Formula I to alleviate alopecia

Thyroid hormone is also known to stimulate hair growth. Synthetic thyroid hormone replacements (i.e., thyromimetics) have also been shown to stimulate hair growth. Such thyromimetics have been described in the literature previously. The reader's attention is directed to European Patent Application No. 1262177, the contents of which are hereby incorporated by reference, for a discussion of such compounds and their use to alleviate alopecia. One particular compound of interest is 2-{4-[3-(4-Fluoro-benzyl)-4-hydroxy-phenoxy]-3,5-dimethyl-phenyl}-2H-[1,2,4]triazine-3,5-dione. Such compounds can be co-administered with the compounds of Formula I to alleviate alopecia.

Anti-androgens can work by a number of different mechanisms. For example, some compounds block the conversion of testosterone to 5-α-dihydrotestosterone, which is responsible for the biological effect in many tissues. 5-Alpha-reductase inhibitors, such as finasteride, have been shown to stimulate hair growth and to decrease sebum production. Finasteride is commercially available from Merck under the trade name Propecia®. Examples of other 5-α-reductase inhibitors include dutasteride, (Glaxo Smithkline). Such compounds can be co-administered with the compounds of Formula I to alleviate alopecia and/or to decrease sebum production.

Protein kinase C inhibitors have also been shown to stimulate hair growth and induce anagen. Calphostin C, which is a selective inhibitor of protein kinase C, has been shown to induce anagen. Other selective protein kinase C inhibitors, such as hexadecylphosphocholine, palmitoyl-DL-carnitine chloride, and polymyxin B sulfate have also been shown to induce anagen. [Skin Pharmacol Appl Skin Physiol 2000 May-August; 13(3-4):133-42]. Any such protein kinase C inhibitor can be co-administered with a compound of Formula I to alleviate alopecia.

Immunophilins are a family of cytoplasmic proteins. Their ligands include cyclosporin and FK506. They are derived from fungi and were developed primarily for their potent immunosuppressive properties. Cyclosporin binds to the proteins, cyclophilins, while FK506 binds to FK binding proteins (FKBPs). All of these compounds have been shown to stimulate hair growth and induce anagen. Any such immunophilin ligands can be co-administered with a compound of Formula I to alleviate alopecia.

Acyl CoA cholesterol acyl transferase (ACAT) inhibitors were initially evaluated for the treatment of elevated serum cholesterol. It was subsequently discovered that these compounds decrease sebum production (U.S. Pat. No. 6,133,326). Any such ACAT inhibitor can be co-administered with a compound of formula I to decrease sebum production, alleviate oily skin, etc.

Antibiotics, such as tetracycline and clindamycin, have been used to alleviate acne. The antibiotic eradicates the microorganism, Propionbacterium acnes, leading to a reduction in the patient's acne. The compounds of Formula I can be co-administered with any antibiotic suitable for the treatment of acne.

Retinoids, such as isotretinoin, have been shown to decrease sebum production and are used to treat acne. These retinoids can be co-administered with a compound of Formula I in order to decrease sebum production and/or to treat acne.

Estrogen and progesterone have each been shown to decrease sebum production. These compounds, or any synthetic agonist of such compounds, may be co-administered with a compound of formula I in order to decrease sebum production.

As used in this application, co-administered refers to administering a compound of Formula I with a second medicinal, typically having a differing mechanism of action, using a dosing regimen that promotes the desired result. This can refer to simultaneous dosing, dosing at different times during a single day, or even dosing on different days. The compounds can be administered separately or can be combined into a single formulation. Techniques for preparing such formulations are described below.

Formulations

If desired, the compounds can be administered directly without any carrier. However, to ease administration, they will typically be formulated into pharmaceutical carriers. Likewise, they will most typically be formulated into dermatological, or cosmetic carriers. In this application the terms “dermatological carrier” and “cosmetic” carrier are being used interchangeably. They refer to formulations designed for administration directly to the skin or hair.

Pharmaceutical and cosmetic compositions can be manufactured utilizing techniques known in the art. Typically an effective amount of the compound will be admixed with a pharmaceutically/cosmetically acceptable carrier.

For oral administration, the compounds can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the compounds of Formula I can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or non-aqueous pharmaceutically acceptable solvent, which may also contain suspending agents, sweetening agents, flavoring agents, and preservative agents as are known in the art.

For parenteral administration the compounds may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, buffers, etc., as are known in the art. When the compounds are being administered intrathecally, they may also be dissolved in cerebrospinal fluid as is known in the art.

The compounds of this invention will typically be administered topically. As used herein, topical refers to application of the compounds (and optional carrier) directly the skin and/or hair. The topical composition according to the present invention can be in the form of solutions, lotions, salves, creams, ointments, liposomes, sprays, gels, foams roller sticks, or any other formulation routinely used in dermatology.

Thus, a further embodiment relates to cosmetic or pharmaceutical compositions, in particular dermatological compositions, which comprise at least one of the compounds corresponding to Formula I above. Such dermatological compositions will contain from 0.001% to 10% w/w% of the compounds in admixture with a dermatologically acceptable carrier, and more typically, from 0.1 to 5 w/w % of the compounds. Such compositions will typically be applied-from 1 to 4 times daily. The reader's attention is directed to Remington's Pharmaceutical Science, Edition 17, Mack Publishing Co., Easton, Pa. for a discussion of how to prepare such formulations.

The compositions according to the invention can also consist of solid preparations constituting cleansing soaps or bars. These compositions are prepared according to the usual methods.

The compounds can also be used for the hair in the form of aqueous, alcoholic or aqueous-alcoholic solutions, or in the form of creams, gels, emulsions or mousses, or alternatively in the form of aerosol compositions also comprising a propellant under pressure. The composition according to the invention can also be a hair care composition, and in particular a shampoo, a hair-setting lotion, a treating lotion, a styling cream or gel, a dye composition, a lotion or gel for preventing hair loss, etc. The amounts of the various constituents in the dermatological compositions according to the invention are those conventionally used in the fields considered.

The medicinal and cosmetics containing the compounds of the invention will typically be packaged for retail distribution (i.e. an article of manufacture). Such articles will be labeled and packaged in a manner to instruct the patient how to use the product. Such instructions will include the condition, which may be treated, duration of treatment, dosing schedule, etc.

The compounds of Formula I may also be admixed with any inert carrier and utilized in laboratory assays in order to determine the concentration of the compounds within the serum, urine, etc., of the patient as is known in the art. The compounds may also be used as a research tool.

Use in Livestock

In addition to the therapeutic and cosmetic uses described above, the compounds may also be used to promote the growth of animals, especially livestock. The compounds will increase the rate at which the animals gain weight, increase the leanness of the resulting meat and improve the efficiency of feed utilization. This may be accomplished by administering an effective amount of a compound of Formula I to an animal receiving adequate nutrition to support growth (i.e. sufficient calories, amino acids, vitamins, minerals, essential fats, etc).

To simplify administration, the compound is typically mixed with animal feeds or prepared in the form of an animal-feed premix, concentrate, or supplement which can be blended with animal feeds. Regardless of the procedure selected, the compound will typically be present at levels of from about 0.05 to 500 ppm in the feed.

Animal-feed premixes, supplements or concentrates can be prepared by mixing on a weight basis about 0.5 to 50% of a compound with about 50 to 99.5% of an edible diluent. Diluents suitable for use in the manufacture of animal-feed supplements, concentrates, and premixes include the following: corn meal, soybean meal, bone meal, alfalfa meal, cottonseed oil meal, urea, molasses, and other similar materials. Use of the diluents in feed supplements, concentrates, and premixes improves uniformity of distribution of the active ingredient in the finished feed.

Feeds for swine, cattle, sheep, fish, and goats typically contains about 0.05 to 400 grams of active ingredient per ton of feed. Poultry and domestic-pet feeds range from about 0.05 to 400 grams per ton of feed.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data is being presented in order to further illustrate the invention. This disclosure should not be construed as limiting the invention in any manner.

Animal Model for Androgenetic Alopeica

As described above, alopecia is a problem that medical science has devoted considerable resources to. As with any disease process, animal models have been developed to allow scientists to screen compounds for their potential relative efficacy. Those compounds showing the greatest efficacy in these animal models are considered for further study in humans.

Two different animal models have been developed to date for hair growth. The first is the telogen conversion assay, which uses female C3H/HeN mice. The second model uses stump-tailed macaques, which are monkeys that suffer from androgenetic alopecia.

The telogen conversion assay measures the potential of a compound to convert mice in the resting stage of the hair growth cycle (“telogen”) to the active stage of the hair growth cycle (“anagen”). This assay takes advantage of the fact that the fur (i.e. hair) of 7 week old C3H/HeN mice is in the telogen phase. This phase continues until about 75 days of age. In this assay, selected areas of the mice are shaved, contacted with a test agent, or a control, and the difference in the rate of hair growth is measured (i.e. induction of the anagen phase). The first sign of anagen is the darkening of skin color as melanocytes in the follicles start to synthesize melanin, in preparation for the production of pigmented hairs. This model has a number of advantages. This includes the ready availability of female CH3HeN mice, the ability to screen large numbers of compounds quickly, and the ease of housing and handling such animals.

The primary disadvantage of this model is its lack of androgenetic dependency. While the exact cause of human baldness is not known, it is well documented that androgens induce a regression of hair follicles in the scalp. This post adolescent regressive change is a fundamental cause of male pattern baldness, (i.e. “androgenetic alopecia). This phenomenon occurs in both men and women who have inherited the genetic trait for alopecia, as mentioned previously. For a more detail discussion of the effects of androgens on human scalps, the reader's attention is directed to Trueb, RM, Molecular Mechanisms of Androgenic Alopecia, Exp. Gerontology, 2002, 27:981-990.

Researchers looked for other animals whose hair growth was similar to that of humans. These lead researchers to stump-tailed macaques. These primates also suffer from androgentic alopecia. Essentially all post adolescent macaques, in both sexes, exhibit the development of baldness. Like the development of male pattern baldness in humans, androgens are an indispensable triggering factor in macaque baldness. Thinning of the frontal scalp hairs begins to appear around the same age (4 years) when serum levels of testosterone become drastically elevated in male animals. Although the elevation of testosterone in females is approximately one tenth that of the male level, there is no difference in the incidence and the age of onset of baldness between male and female stump-tailed macaques. Topical application of anti-androgens have reversed this baldness in animals of both sexes. Pan, H J et al, Evaluation of RU58841 as an anti-androgen in prostate PC3 cells and a topical anti-alopecia agent in the bald scalp of stump tailed macaques. Endocrine 1998; 9:39-43.)

While this model is a significant improvement over the telogen conversion assay as a model for human baldness, it suffers from a number of practical disadvantages. The macaques are expensive, relatively rare, labor intensive to maintain, and require long wash out periods between testing. Thus, the macaque is not a practical model for screening large numbers of compounds

It has been discovered that male C3H/HeN mice may be used in the telogen conversion assay, when evaluating anti-androgen test compounds. Thus, the model relates to a modification of the existing telogen conversion assay. Male C3H/HeN mice approximately 7 weeks old are utilized. These animals are also uniformly in telogen, like their female counterparts. However, once shaven, the androgens inherently present in these male mice inhibit the conversion of the hair follicles to the anagen phase. An anti-androgen will block this androgenic effect and the follicles will convert to anagen, like their female counterparts.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data is being presented in order to further illustrate the invention. This disclosure should not be construed as limiting the invention in any manner.

### EXAMPLE 1

(R)-(+)-6-[Methyl-(1-Phenyl-ethyl)-amino]-4-trifluoromethyl-nicotinonitrile 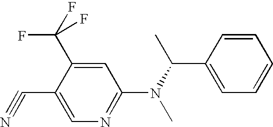
Step A) Amination 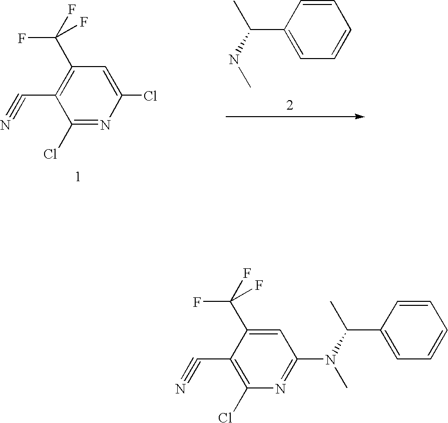
The starting materials, 2,6-dichloro-4-trifluoromethyl-nicotinonitrile (5.0 g, 21 mmol) and (R)-(+)-N,alpha-dimethyl benzylamine (2.6 g, 19 mmol) were dissolved in 85 ml of dry DMF, then K₂CO₃ (5.0 g) was suspended in the mixture, the reaction was stirred at 91° C. for 1 h, it was checked by MS. When it was completed, the K₂CO₃ was filtered out, the solvent was removed to get an oily liquid, it was on vacuum overnight, the crude product was a slight yellow solid. The crude product was washed with hot hexane to yield a pure product (6.16 g, 96.16%).

MS: 340.1 (M+1 for C₁₆H₁₃Cl N₃F₃) LCMS: C-18 Column (25% H₂O/75%CH₃CN), Ret. Time: 1.99 min Purity: 99.1%,
Step B: Dehalogenation

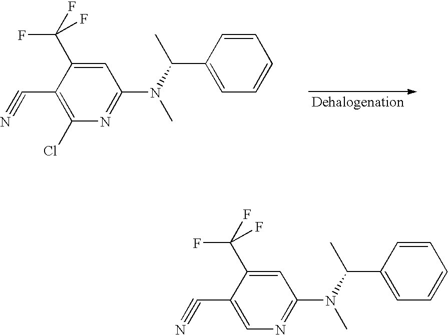

6.16 g of the product of Step A was dissolved in 90 ml of THF, then 10 ml of triethyl amine and 1.0 g 10% Pd/C catalyst were added at 0.0 h. The reaction was run at high-pressure (hydrogen gas) for 0.1 h. The catalyst was removed by filtration. The crude product was purified by column using Hexane: Ethyl acetate=5:1 as elute to yield (R)-(+)-6-[Methyl-(1-Phenyl-ethyl)-amino]-4-trifluoromethyl-nicotinonitrile, as white crystals (5.20 g)

MS: 306.1 (M+1 for C₁₆H₁₄N₃F₃), LCMS: C-18 Column (25% H₂O/75% CH₃CN), Ret. Time: 1.74 min Purity: 99.8%, M.P=47.2-47.7° C.

### EXAMPLES 2-12

Using the general procedure of Example 1, but substituting the relevant starting materials, the compounds described in Table I were prepared. The chromatograph was performed on a Foxy 200 fraction collector, using pre-prepared Biotage Silicon Gel column, Hexane:ethyl aceate=5:1 as the elute solvent. The mass spectra in Table I were recorded with an HP 1100-MSD LC/MS spectrometer by using 50% CH3CN:50% H2O as the solvent.

TABLE 1

| Example | Structure | Name | RT | Base Peak |
|---|---|---|---|---|
| 2 | 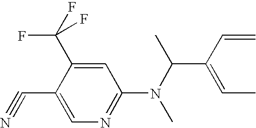 | 6-[Methyl-(1-Phenyl- ethyl)-amino]-4- trifluoromethyl- nicotinonitrile | 1.75 | MS: 306.1 (M + 1 for C₁₆H₁₄N₃F₃), |
| 3 | 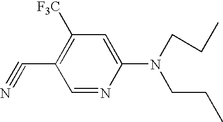 | 6-Dipropylamino-4- trifluoromethyl- nicotinonitrile | 3.42 | MS: 272.1 (M + 1 for C₁₃H₁₆N₃F₃), |
| 4 | 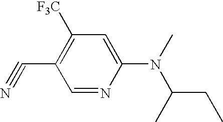 | 6-(sec-Butyl-methyl- amino)-4-trifluoromethyl- nicotinonitrile | 3.56 | MS: 258.1 (M + 1 for C₁₂H₁₄N₃F₃ |
| 5 | 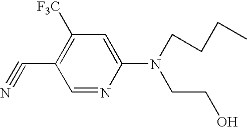 | 6-[Butyl-(2-hydroxy- ethyl)-amino]-4- trifluoromethyl- nicotinonitrile | | MS: 290.1 (M + 1 for C₁₃H₁₆N₃F₃O) |
| 6 | 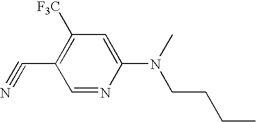 | 6-(Butyl-methyl-amino)-4- trifluoromethyl- nicotinonitrile | 1.55 | MS: 258.1 (M + 1 for C₁₂H₁₄N₃F₃), |
| 7 | 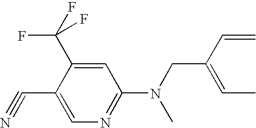 | 6-(Benzyl-methyl-amino)- 4-trifluoromethyl- nicotinonitrile | 1.40 | MS: 292.1 (M + 1 for C₁₅H₁₂N₃F₃), |
| 8 | 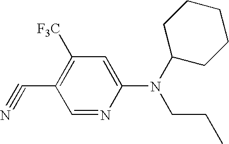 | 6-(Cyclohexyl-propyl- amino)-4-trifluoromethyl- nicotinonitrile | 2.95 | MS: 312.5 (M + 1 for C₁₆H₂₀N₃F₃), |
| 9 | 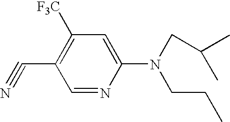 | 6-(Cyclopropylmethyl- propyl-amino)-4- trifluoromethyl- nicotinonitrile | 2.10 | MS: 284.1 (M + 1 for C₁₄H₁₆N₃F₃), |
| 10 | 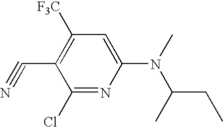 | 6-(sec-Butyl-methyl- amino)-)-2-chloro-4- trifluoromethyl- nicotinonitrile | 4.21 | MS: 292.1 (M + 1 for C₁₂H₁₃ClN₃F₃), |
| 11 | 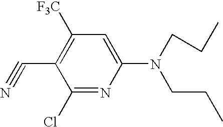 | 6-Dipropylamino-2-chloro- 4-rtifluoromethyl- nicotinonitrile | 4.51 | MS: 306.1 (M + 1 for C₁₃H₁₅ClN₃F₃), |
| 12 | 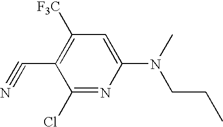 | 2-Chloro-6-(methyl- propyl-amino)-4- trifluoromethyl- nicotinonitrile | 1.83 | MS: 278.0 (M + 1 for C₁₁H₁₁ClN₃F₃) |

### Example 13

The compounds of Formula I have affinity for the androgen receptor. This affinity has been demonstrated for selected compounds using the human receptor. The description below describes how the assay was carried out.

Competitive binding analysis was performed on baculovirus/Sf9 generated hAR extracts in the presence or absence of different concentrations of test agent and a fixed concentration of ³H-dihydrotestosterone (³H-DHT) as tracer. This binding assay method is a modification of a protocol previously described (Liao S., et. al. J. Steroid Biochem. 20:11-17 1984). Briefly, progressively decreasing concentrations of compounds are incubated in the presence of hAR extract (Chang et al. P.N.A.S. Vol. 89, pp. 5546-5950, 1992), hydroxylapatite, and 1 nM³ H-DHT for one hour at 4° C. Subsequently, the binding reactions are washed three times to completely remove excess unbound ³H-DHT. hAR bound ³H-DHT levels are determined in the presence of compounds (=i.e competitive binding) and compared to levels bound when no competitor is present (=i.e. maximum binding). Compound binding affinity to the hAR is expressed as the concentration of compound at which one half of the maximum binding is inhibited. Table II below provides the results that were obtained for selected compounds (reported data is the mean of repeated individual tests as shown below)

| | | AR Binding |
|---|---|---|
| Example # | Structure | IC₅₀ (nM) |
| 1 | 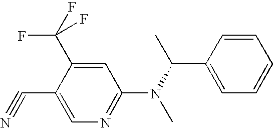 | 20 (c) |
| 2 | 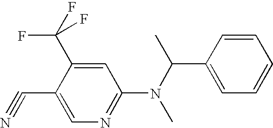 | 123 (a) |
| 3 | 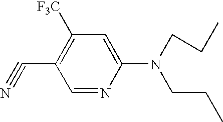 | 22 (a) |
| 4 | 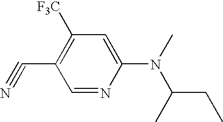 | 10 (a) |
| 5 | 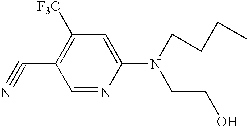 | 548 (a) |
| 6 | 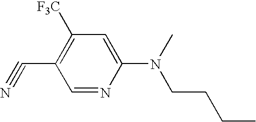 | 149 (d) |
| 7 | 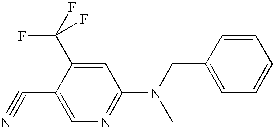 | 538 (a) |
| 8 | 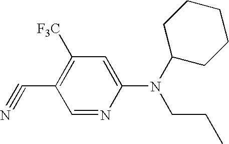 | 425 (a) |
| 9 | 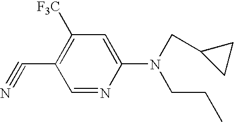 | 0.79 (a) |
| 10 | 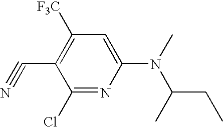 | 0.5 (a) |
| 11 | 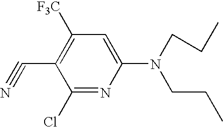 | 8.2 (a) |
| 12 | 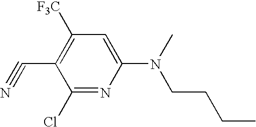 | 88 (a) |

A—mean of two tests
B—mean of three tests
C—mean of ten tests
D—mean of four tests

### Example 14

The compounds ability to antagonize the effects of androgen on the androgen receptor were determined in a whole cell assay as described immediately below.

Experimental Procedure for AR Antagonist Cell Assay

Cell line: MDA-MB453-MMTV clone 54-19. This cell line is a stable transfected cell line with MDA-MB453 cell background (a human breast tumor cell line expressing androgen receptor). A MMTV minimal promoter containing ARE was first cloned in front of a firefly luciferase reporter gene. Then the cascade was cloned into transfection vector pUV120puro. Electroporation method was used for transfecting MDA-MB-453 cell. Puromycin resistant stable cell line was selected.

Cell Culture Media and Reagents:

Culture medium: DMEM (high glucose, Gibco cat #:11960-044), 10% FBS, and 1% L-glutamine

Plating medium: DMEM (phenol red free), 10% charcoal treated HyClone serum, 1% L-glutamine

Assay medium: DMEM (phenol red free), 1% charcoal treated HyClone serum, 1% L-glutamine, and 1% penicillin/streptomycin

3× luciferase buffer: 2% beta-mercaptoethanol, 0.6% ATP, 0.0135% luciferine in cell lysis buffer

Assay Procedure:

1. Cells are maintained in culture medium, splitting cells when they reach 80-90% confluence
2. To test compounds, 10,000 cells/well are plated to opaque 96 cell culture plate in 100 uvwell plating medium, culture for overnight at 37° C. in cell culture incubator
3. Carefully remove plating medium, then add 80 ul/well of pre-warmed assay medium, add 10 uvwell testing compound (final concentration at) 1000 nM, 200 nM, 40 nM, 8 nM, 1.6 nM, and 0.32 nM), incubate at 37° C. for 30 minutes
4. Add 10 ul/well freshly prepared DHT (final concentration at 100 pM) to each well, incubate at 37° C. for 17 hr (overnight)
5. Add 50 ul/well 3× luciferase buffer, incubate at room temperature for 5 minutes, then count on Luminometer
   The fold induction over background by 100 pM DHT in the absence of testing compounds is standardized as 100% and experimental result is expressed as percentage of inhibition by testing compounds.

The results are described below in Table III. The results are reported as the mean of multiple tests as described below (the numbers of tests are indicated in the footnote). N.D. denotes that the compound was not tested.

| Example | | AR Cell |
|---|---|---|
| # | Structure | IC50 (nM) |
| 1 | 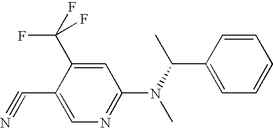 | 45 (a) |
| 2 | 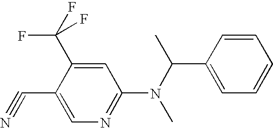 | 229 (a) |
| 3 | 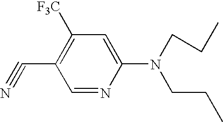 | >1000 (b) |
| 4 | 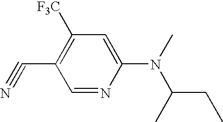 | >1000 (d) |
| 5 | 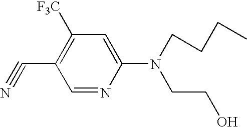 | N.D. |
| 6 | 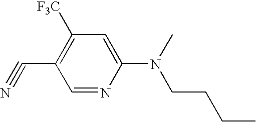 | >1000 (c) |
| 7 | 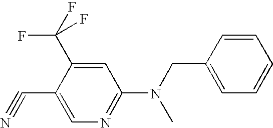 | N.D. |
| 8 | 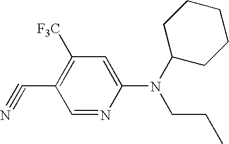 | N.D. |
| 9 | 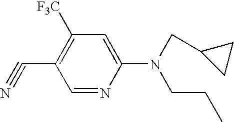 | >1000 (a) |
| 10 | 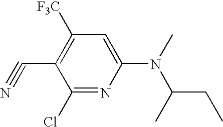 | >1000 (b) |
| 11 | 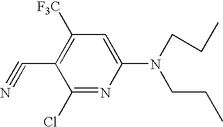 | >1000 (b) |
| 12 | 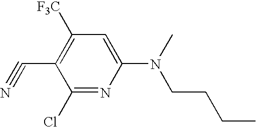 | 587 (a) |

A—mean of two tests
B—mean of three tests
C—mean of four tests
D—mean of five tests

### Example XV

The compound described in Example I was submitted for further testing utilizing the modified telogen conversion assay, described above. The testing was carried out in the following manner.

Male C3H/HeN mice, 6 to 7 weeks old (Charles River Laboratories, Raleigh, N.C.) were used for the study. Fur was clipped from the dorsal region of the mice prior to initiation of the study. Only mice with pink skin, a visual indication of the telogen phase, were selected for inclusion in the study.

The test compound was dissolved in a vehicle consisting of propylene glycol (30%) and ethanol (70%) to achieve a concentration of 3% w/v. It was applied topically to the clipped dorsal region of the mice in one test group (7-10 mice) in a volume of 20 μl/cm². A second group of animals received only the vehicle to as a control. Treatments were applied twice daily for 4 weeks.

The treatment area was observed and graded every other day for signs of hair growth. The hair growth response was quantified by recording, for each animal, the day on which signs of hair growth first appeared over the treated area. The first sign of anagen was the darkening of skin color as melanocytes in the follicles started to synthesize melanin in preparation for the production of pigmented hairs. The mice were observed for 35 days or longer. The percentage of mice showing signs of hair growth in both the treatment group and the control group is graphically depicted below in Figure I. The compound of Example 1 produced substantial hair growth by stimulating the induction of the anagen in the test animals.

1. A compound of the formula: 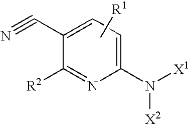
or a pharmaceutically acceptable salts, thereof, wherein;
a) R¹ is represented by, (C₁-C₂)alkyl, substituted with one or more halogens, or (C₁-C₂)alkoxy, substituted with one or more halogens,
b) R² is represented by hydrogen or halogen,
c) X¹ and X² are each independently represented by
i) (C₁-C₁₂)alkyl, optionally substituted,
ii) (C₂-C₁₂)alkenyl, optionally substituted,
iii) (C₂-C₁₂)alkynyl, optionally substituted,
iv) (C₃-C₁₀)cycloalkyl, optionally substituted,
v) (C₃-C₁₀)cycloalkyl(C₁-C₆)alkyl, in which the alkyl and cycloalkyl moieties may each be optionally substituted,
vi) (C₆-C₁₀)aryl, optionally substituted,
vii) (C₆-C₁₀)aryl(C₁-C₆)alkyl, in which both the alkyl and aryl moieties may be optionally substituted,
viii) —(CH₂)_{q}—CH₂—ZH, in which Z is S or O and q is an integer from 1-11,
ix) —(CH₂)ₙ—Y—(CH₂)ₚ—CH₃, in which Y is O or S, n is an integer from 1 to 4, and p is an integer from 1 to 4,
x) —[CH₂]ₘ—C(O)R³, in which m is an integer selected from 1 to 8 and R³ is represented by hydrogen, (C₁-C₁₂)alkyl, (C₆-C₁₀)aryl, or (C₆-C₁₀)aryl(C₁-C₆)alkyl, in which both the alkyl and aryl moieties may be optionally substituted,
xi) —[CH₂]ₘ—C(O)—O—R⁴, in which m is as defined above and R⁴ is represented by hydrogen, (C₁-C₁₂)alkyl, (C₆-C₁₀)aryl, or (C₆-C₁₀)aryl(C₁-C₆)alkyl, in which the alkyl and aryl moieties may be optionally substituted,
xii) —[CH₂]m—C(O)—NR⁵R⁶ in which m is as described above, and R⁵ and R⁶ are each independently represented by hydrogen, (C₁-C₁₂)alkyl, (C₆-C₁₀)aryl, or (C₆-C₁₀)aryl(C₁-C₆)alkyl, in which the alkyl and aryl moieties may each be optionally substituted,
i) heteroaryl, optionally substituted,
ii) heteroaryl(C₁-C₆)alkyl, in which the heteroaryl and alkyl moieties may each be optionally substituted,
iii) heterocyclic, optionally substituted, or,
iv) heterocyclic(C₁-C₆)alkyl, in which the alkyl and heterocyclic moieties may each be optionally substituted.

2. A compound according to claim 1 in which R¹ is represented by trifluomethyl.

3. A compound according to claim 2 in which said trifluoromethyl is located at the 4-position of the pyridine ring.

4. A compound according to claim 2 in which R² is hydrogen.

5. A compound according to claim 2 in which X¹ is (C₁-C₁₂)alkyl and X² is (C₆-C₁₀)aryl(C₁-C₆)alkyl.

6. A compound according to claim 2 in which X¹ and X² are each (C₁-C₁₂)alkyl

7. A compound according to claim 2 in which X¹ is (C₁-C₁₂)alkyl and X² is (C₃-C₁₀)cycloalkyl(C₁-C₆)alkyl.

8. A compound according to claim 1 selected from the group consisting of
(R)-(+)-6-[Methyl-(1-Phenyl-ethyl)-amino]-4-trifluoromethyl-nicotinonitrile,
(R)-(+)-2-Chloro-6-[methyl-(1-phenyl-ethyl)-amino]-4-trifluoromethyl-nicotinonitrile,
6-[methyl-(1-phenyl-ethyl)-amino]-4-trifluoromethyl-nicotinonitrile,
6-[methyl-(1-phenyl-ethyl)-amino]-4-trifluoromethoxy-nicotinonitrile,
6-[methyl-(1-(4-fluorophenyl)-ethyl)-amino]-4-trifluoromethyl-nicotinonitrile,
6-[methyl-(1-(3-hydroxyphenyl)-ethyl)-amino]-4-trifluoromethyl-nicotinonitrile,
6-[butyl(1-(3-hydroxyphenyl)-ethyl)-amino]-4-trifluoromethoxy-nicotinonitrile,
6-dipropylamino-4-trifluoromethyl-nicotinonitrile,
2-chloro-6-dimethylamino-4-trifluoromethyl-nicotinonitrile,
6-(hexyl-octyl-amino)-4-trifluoromethyl-nicotinonitrile,
6-(sec-butyl-methyl-amino)-4-trifluoromethyl-nicotinonitrile,
6-[butyl-(2-hydroxy-ethyl)-amino]-4-trifluoromethyl-nicotinonitrile,
6-(butyl-methyl-amino)-4-trifluoromethyl-nicotinonitrile,
6-(benzyl-methyl-amino)-4-trifluoromethyl-nicotinonitrile,
6-(cyclohexyl-propyl-amino)-4-trifluoromethyl-nicotinonitrile,
6-(cyclopropylmethyl-propyl-amino)-4-trifluoromethyl-nicotinonitrile,
6-(sec-butyl-methyl-amino)-2-chloro-4-trifluoromethyl-nicotinonitrile,
6-Dipropylamino-2-chloro-4-trifluoromethyl-nicotinonitrile,
6-(propyl-methyl-amino)-2-chloro-4-trifluoromethyl-nicotinonitrile, and,
6-(Butyl-methyl-amino)-2-chloro-4-trifluoromethyl-nicotinonitrile.

9. Use of a compound according to claim 1 as a medicine.

10. A method for inhibiting activation of the androgen receptor, comprising, administering a compound according to claim 1 to a patient in need thereof.

11. A pharmaceutical composition comprising a compound according to claim 1 in admixture with 1, or more, pharmaceutically acceptable excipients.

12. A topical pharmaceutical formulation comprising a compound according to claim 1 in admixture with 1, or more, pharmaceutically acceptable excipients suitable for dermal application.

13. A kit comprising a compound according to claim 1 packaged for retail distribution which advises a consumer how to utilize the compound to alleviate a condition selected from the group consisting of acne, alopecia, and oily skin.

14. A method of treating a condition selected from the group consisting of hormone dependent cancers, benign hyperplasia of the prostate, acne, hirsutism, excess sebum, alopecia, premenstrual syndrome, lung cancer, precocious puberty, osteoporosis, hypogonadism, age-related decrease in muscle mass, and anemia comprising the administration of a compound according to claim 1.
